(19) United States
(12) Patent Application Publication
(10) Pub. No.: US 2004/0053215 A1
(43) Pub. Date: Mar. 18, 2004

(54) Method and primers for detecting viral genes

(76) Inventors: Joseph Ian Cooper, Oxford (GB); Martin Naylor, Oxford (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/380,565

(22) PCT Filed: Sep. 10, 2001
(86) PCT No.: PCT/GB01/04053

(30) Foreign Application Priority Data
Sep. 14, 2000 (GB) 0022505.2

(51) Int. Cl. C12Q 1/70

(52) U.S. Cl. 435/5

(57) ABSTRACT

A method for detecting luteoviruses comprises extracting nucleic acid from plant tissue and making a first strand DNA using a PCR primer and amplifying the DNA using a second PCR primer to obtain as PCR product and comparing amino acid sequences in the PCR product obtained with known amino acid sequences characteristic of the luteovirus to detect the presence of the luteovirus.

### TECHNICAL FIELD

The present invention relates to a method for the detection of luteoviruses using polymerase chain reaction (PCR) primers and to amino acid sequences which can be used to design the PCR primers. In particular, the invention relates to methods and primers for detection of carrot red leaf luteovirus (CRLV).

### PRIOR ART

Carrot motley dwarf is a serious viral disease of carrots and other umbelliferous plants such as parsley and chervil. Symptoms include red and yellow discolouration of leaves and stunting. In general, luteoviruses can be spread efficiently by aphids, cause serious disease in vegetables and cereals and are of global economic importance.

Carrot motley dwarf (CMD) is an aphid transmissible virus disease caused by the combination of the two types of virus (carrot red leaf luteovirus (CRLV) and carrot mottle umbravirus (CMoV)). Although each of these viruses are independently pathogenic (Watson, M., Serjeant, E. P., and Lennon, E. A. (1964) Annals of Applied Biology 54;153-166), the umbravirus component can only be transmitted by aphids when encapsidated by luteovirus-coded coat protein (Elnagar, S., and Murant, A. F. (1978) Annals of Applied Biology 89, 237-244.; Waterhouse, P. M., and Murant, A. F. (1983) Annals of Applied Biology 103;455-464). Luteoviruses are largely phloem restricted (Murant, A. F., Roberts, I. M. (1979). Virus-like particles in phloem tissue of chervil (Anthriscus cerefolium) infected with carrot red leaf luteovirus. Annals of Applied Biology 92;343-346) whereas umbraviruses are able to spread systemically throughout the plant. Luteoviruses are able to gain from association with CMoV because CMoV facilitates luteovirus accumulation (Barker, H. (1989) Ann.appl.Biol 115;71-78), and though each can persist alone, the combination has a potential for greater fitness. Other examples of luteoviruses include potato leaf roll virus (PLRV), beet western yellows virus (BWYV) and barley yellow dwarf virus (BYDV).

There is to date no rapid and sensitive detection technique for CMD viruses. It is thought that this could be due to several reasons, firstly, the carrot mottle component of CMD cannot easily be detected serologically as the virus has no coat protein. In addition, a high level of genetic variation is proposed to exist among carrot mottle virus isolates, making molecular detection difficult. The carrot red leaf component of CMD can be detected serologically, but the virus accumulates only in the phloem of infected plants and only at low levels.

Robertson, N. L. French, R.Gray S. M (1991) J. Gen. Virol. 72:1473-1477 disclose a PCR based method using a single luteovirus primer pair to diagnose a range of luteoviruses including five barley yellow dwarf luteovirus serotypes by one assay using the luteoviral coat protein gene sequences to design PCR primers. However the primer pairs disclosed in this document are unable to detect CRLV.

Vercruysse et al (2000, J. Virological Methods 88:153-161) describes the design of a pair of redundant primers designed to target conserved regions of the RdRp genes from an unpublished partial sequence of a Canadian isolate of CRLV and sequences from five potato leafroll polerovirus (PLRV) isolates. The primers were able to identify cDNA from CRLV and PLRV. However, the primers disclosed by this document were not able to detect luteoviruses other than CRLV and PLRV.

Therefore, although there exists a diagnostic assay which is able to identify CRLV infection, there is a need for a single broad spectrum diagnostic assay which is capable of diagnosing a large number of luteoviruses including the agronomically important CRLV. Thus it can be seen that a luteovirus detection system capable of detecting a broad spectrum of luteoviruses including CRLV would provide a contribution to the art.

### DISCLOSURE OF THE INVENTION

The present inventors have now devised primers capable of detecting the presence of a number of luteoviruses including CRLV sequence. In particular, a previously unpublished CRLV nucleic acid sequence has been aligned with nucleic acid sequences of a number of other luteoviruses and primers have been designed to conserved regions of the sequence corresponding to regions of high degeneracy in the translated amino acid sequence.

Accordingly, there is provided in a first aspect of the present invention, a primer capable of hybridising to conserved regions of nucleic acid of carrot red leaf luteovirus (CRLV), potato leafroll polerovirus (PLRV) and at least one of barley yellow dwarf virus (BYDV) and beet western yellow virus (BWYV). In preferred embodiments, the primer is capable of hybridising to conserved regions of nucleic acid of carrot red leaf luteovirus (CRLV), potato leafroll polerovirus (PLRV), barley yellow dwarf virus (BYDV) and beet western yellow virus (BWYV).

Oligonucleotide primers may be designed on the basis of amino acid sequence information, taking into account the degeneracy of the genetic code, and, where appropriate, codon usage of the organism from which the candidate nucleic acid is derived. An oligonucleotide for use in nucleic acid amplification may have about 10 or fewer codons (e.g. 6, 7 or 8), i.e. be about 30 or fewer nucleotides in length (e.g. 18, 21 or 24). Generally specific primers are upwards of 14 nucleotides in length, but need not be longer than 18-20. Those skilled in the art are well versed in the design of primers for use in processes such as PCR. Various techniques for synthesizing oligonucleotide primers are well known in the art, including phosphotriester and phosphodiester synthesis methods.

Preferred amino acid sequences suitable for use in the design of probes or PCR primers may include conserved (completely, substantially or partly) regions of sequences corresponding to a RNA dependent RNA polymerase (RdRp). In one embodiment, the PCR primers which can be used can be designed by obtaining the nucleotide sequence corresponding to a RNA dependent RNA polymerase (RdRp) gene in an isolate of CRLV, making alignments of putative RNA-dependent RNA polymerase (RdRp) proteins of BWYV, PLRV and BYDV, and the predicted amino acid sequence of CRLV and using the regions of amino acid conservation to design PCR primers.

The CRLV sequence is preferably all or part of the CRLV sequence of FIG. 2.

Such sequences include the FVKGEPH, GFKVEV, EDDMEV or KGEPHK motifs of the CLRV amino acid sequence of FIG. 2.

Thus preferred primers according to the invention include primers which encode the amino acid sequence FVKGEPH (SEQ ID NO:1) (5′ TTY/GTN/AAR/GGN/GAR/CCN/CAY 3′ (CL1, SEQ ID NO:2)); primers which hybridise to the nucleic acid encoding amino acid sequence GFKVEV (SEQ ID NO:3) (5′NAC/YTC/NAC/YTT/RAA/NCC 3′ (CL2, SEQ ID NO:4)) and primers which hybridise to the nucleic acid encoding amino acid sequence EDDMEV (SEQ ID NO:5) (5′ CK/NAC/YTC/CAT/RTC/RTC/YTC 3′ (CL3, SEQ ID NO:6)). As an alternative to the CL1 primer disclosed above, a primer which encodes the amino acid sequence KGEPHK (SEQ ID NO:7) (5′ AAR/GGN/GAR/CCN/CA Y/AAR/C 3′ (CL4, SEQ ID NO:8)) may also be used.

In the context of the present invention, “Y”, “R”, “N” and “K” take their normal meanings i.e. “Y”=T/U or C, “R”=G or A, “N”=A or G or C or T/U and “K”=G or T/U.

Uses of Primers

Primers of the invention may be used to diagnose the presence of a luteovirus sequence in a target nucleic acid sample.

Thus in a further aspect of the invention, there is provided a method for diagnosing the presence of a luteovirus sequence in a plant tissue nucleic acid sample, the method comprising the step of: hybridising a primer according to the invention to the nucleic acid.

A luteovirus nucleic acid sequence identified using a method of the invention may be probed under conditions for selective hybridisation and/or subjected to a specific nucleic acid amplification reaction such as the polymerase chain reaction (PCR) (reviewed for instance in “PCR protocols; A Guide to Methods and Applications”, Eds. Innis et al, 1990, Academic Press, New York, Mullis et al, Cold Spring Harbor Symp. Quant. Biol., 51:263, (1987), Ehrlich (ed), PCR technology, Stockton Press, NY, 1989, and Ehrlich et al, Science, 252:1643-1650, (1991)). PCR comprises steps of denaturation of template nucleic acid (if double-stranded), annealing of primer to target, and polymerisation. The nucleic acid probed or used as template in the amplification reaction may be genomic DNA, cDNA or RNA. Other specific nucleic acid amplification techniques include strand displacement activation, the QB replicase system, the repair chain reaction, the ligase chain reaction and ligation activated transcription. For convenience, and because it is generally preferred, the term PCR is used herein in contexts where other nucleic acid amplification techniques may be applied by those skilled in the art. Unless the context requires otherwise, reference to PCR should be taken to cover use of any suitable nucleic amplification reaction available in the art.

A method may include hybridisation of one or more (e.g. two) probes or primers to target nucleic acid. Where the nucleic acid is double-stranded DNA, hybridisation will generally be preceded by denaturation to produce single-stranded DNA. The hybridisation may be as part of a PCR procedure, or as part of a probing procedure not involving PCR. An example procedure would be a combination of PCR and low stringency hybridisation. A screening procedure, chosen from the many available to those skilled in the art, is used to identify successful hybridisation events and isolated hybridised nucleic acid.

Binding of a probe or primer to target nucleic acid (e.g. DNA) may be measured using any of a variety of techniques at the disposal of those skilled in the art. For instance, probes may be radioactively, fluorescently or enzymatically labelled. Other methods not employing labelling of probe include examination of restriction fragment length polymorphisms, amplification using PCR, RN'ase cleavage and allele specific oligonucleotide probing. Probing may employ the standard Southern blotting technique. For instance DNA may be extracted from cells and digested with different restriction enzymes. Restriction fragments may then be separated by electrophoresis on an agarose gel, before denaturation and transfer to a nitrocellulose filter. Labelled probe may be hybridised to the DNA fragments on the filter and binding determined. DNA for probing may be prepared from RNA preparations from cells.

In a preferred embodiment of the invention, the method comprises the steps of treating nucleic acid with a first PCR primer of the invention to obtain a DNA product; treating the DNA product with one or more second PCR primers according to the invention to obtain a PCR product; and comparing the nucleotide sequence or corresponding amino acid sequence of the PCR product with a known nucleotide sequence or corresponding amino acid sequence characteristic of the luteovirus.

In one embodiment of the invention, RT-PCR is carried out using CL2 as a cDNA primer and a mixture of CL1 and CL2 as PCR primers. This set of primers is capable of detecting a broad range of luteoviruses including PLRV, CRLV, beet western yellows virus (BWYV) and beet mild yellowing virus (BMYV).

Where a sample is identified as comprising a luteovirus sequence using the method of the invention, PLRV and CRLV may be discriminated from other luteoviruses such as BWYV and BMYV by repeating the method of the invention, but using CL1 and CL3 as the PCR primers. If luteovirus is detected using CL2 as the cDNA primer and a mixture of CL1 and CL3 as PCR primers, the luteovirus must be CRLV or PLRV.

Thus, in a further aspect of the present invention, there is provided a method for diagnosing the presence of at least one of CRLV or PLRV sequences in a plant tissue, the method comprising the steps of:

providing nucleic acid from plant tissue;

treating the nucleic acid with a PCR primer CL2 to obtain a DNA product;

treating the DNA product with PCR primers CL1 and CL3 to obtain a PCR product; and

comparing a nucleotide sequence or corresponding amino acid sequence in the PCR product with a nucleotide sequence or corresponding amino acid sequence characteristic of the luteovirus to detect the presence of the luteovirus.

The luteovirus sequences may be present due to pathological infection of the plant or may be present as a transgene introduced by recombinant DNA technology. Luteovirus sequences have been introduced as transgenes for crop protection (for example see WO 9418336, Monsanto).

Kits

A primer or primers according to the invention may be provided as part of a kit, e.g. in a suitable container such as a vial in which the contents are protected from the external environment. The kit may include instructions for use of the nucleic acid, e.g. in PCR and/or a method for determining the presence of nucleic acid of interest in a test sample. A kit wherein the nucleic acid is intended for use in PCR may include one or more other reagents required for the reaction, such as polymerase, nucleosides, buffer solution etc. The nucleic acid may be labelled. A kit for use in determining the presence or absence of nucleic acid of interest may include one or more articles and/or reagents for performance of the method, such as means for providing the test sample itself. Such kits form another aspect of the present invention.

In preferred embodiments of the invention the kit includes one or more primers for the determination of the presence of the umbravirus CMoV. Preferred CMoV primers include the carmovirus related primers Carmo-2 and Carmo-6, the CMoV specific primer NC1 or the CMoV specific primer NC-2. Indeed such primers form an independent aspect of the present invention.

The carmovirus related primer Carmo-6 is based on the amino acid sequence VCDDGNN (SEQ ID NO:11); the deduced nucleic acid sequence is:

G(AC)(AC)CTGCAG(AGCT)AC(AG)CA(AG)TC(AG)TC(AGCT)CC(AG)TT(AG)TT (SEQ ID NO:12).

Carmovirus related primer Carmo-2 is based on the amino acid sequence V/PRT/VIQP (SEQ ID NO:13); the deduced nucleic acid sequence is:

| | |
|---|---|
| AA (AG) GTCGACCCG (AT) (AGCT) CC (AGCT) (AC) G (AGCT) GT (AGCT) AT (ACT) CA ACC. | (SEQ ID NO:14) |

The carrot mottle specific primers NC1 and NC2 are based on the amino acid sequences AKGFNAFQ (SEQ ID NO:15) and KYKVKG (SEQ ID NO:16) respectively; the deduced nucleic acid sequences being GC(AGCT)AA(AG)GG(AGCT)TT(CT)AATGC(AGCT)TT(CT)C (SEQ ID NO:17) for NC1 and (AG)CA(AGCT)CC(CT)TT(AGCT)AC(CT)TT(AG)TA(CT)TT (SEQ ID NO:18) for NC2.

The invention will now be further described with reference to the following non-limiting Figures and Examples. Other embodiments of the invention will occur to those skilled in the art in the light of these.

### FIGURES

FIG. 1. shows the nucleotide sequence of CRLV cDNA (SEQ ID NO:9) which includes viral RdRp gene sequence.

FIG. 2 shows the predicted amino acid sequence of CRLV cDNA corresponding to the viral RdRp protein (SEQ ID NO:10)(a translation of part of CRLV sequence shown in FIG. 1).

FIG. 3a Shows a typical alignment of predicted amino acid sequence of CRLV cDNA shown in FIG. 2 with the predicted amino acid sequence of the RdRp proteins (replicase proteins) of Beet Western Yellows Virus (BWYV)(Accession no: P09507), Potato Leafroll Virus (PLRV)(Accession no:P17520), Pea Enation Mosaic Virus (PEMV)(Accession no:P29154) and Southern Bean Mosaic Virus (SBMV)(Accession no:P21405) The amino acid sequences used to design the degenerate RT -PCR primers are shown underlined.

FIG. 3b shows another typical alignment of the predicted amino acid sequence of CRLV cDNA shown in FIG. 2 with the predicted amino acid sequence of the RdRp proteins of Barley Yellow Dwarf Virus (isolate MAV-PS1)(BYDV-MAV-PS1)(Accession no:P29044), Barley Yellow Dwarf Virus (isolate P-PAV) (BYDV-P-PAV) (Accession no:p29045), Potato Leafroll Virus (PLRV-1) (Accession no:P17520), Potato Leafroll Virus (Wageningen Strain) (PLRV-WAG)(Accession no: P11623), Potato Leafroll Virus (Canadian isolate) (PLRV-CA) (Accession no:D00734), Beet Western Yellows Virus (Isolate FL-1) (BWYV-FL1) (Accession no: P09507), Beet Mild Yellowing Virus (BMYV), Barley Yellow Dwarf Virus (NY-RPV isolate) (BYDV-NY-RPV)(Accession no:L25299) and CRLV.

FIG. 4 shows the results of RT-PCR detection of CRLV in plant samples using primers CL1/CL2/CL3.

FIG. 5 shows the results of a CRLV RT-PCR sensitivity assay.

FIG. 6 shows the results of RT-PCR detection of CMoV in plant samples using primers NC1 and NC2.

### EXAMPLES

### Example 1—Design of Degenerate Primers (CRLV)

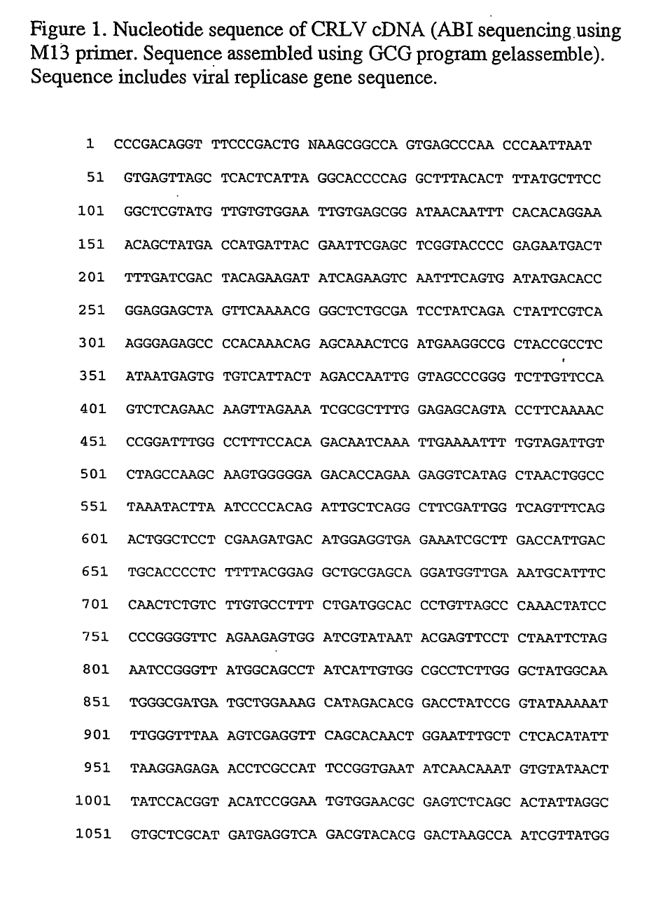

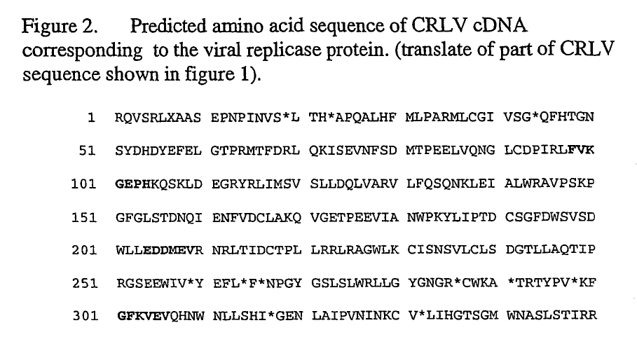

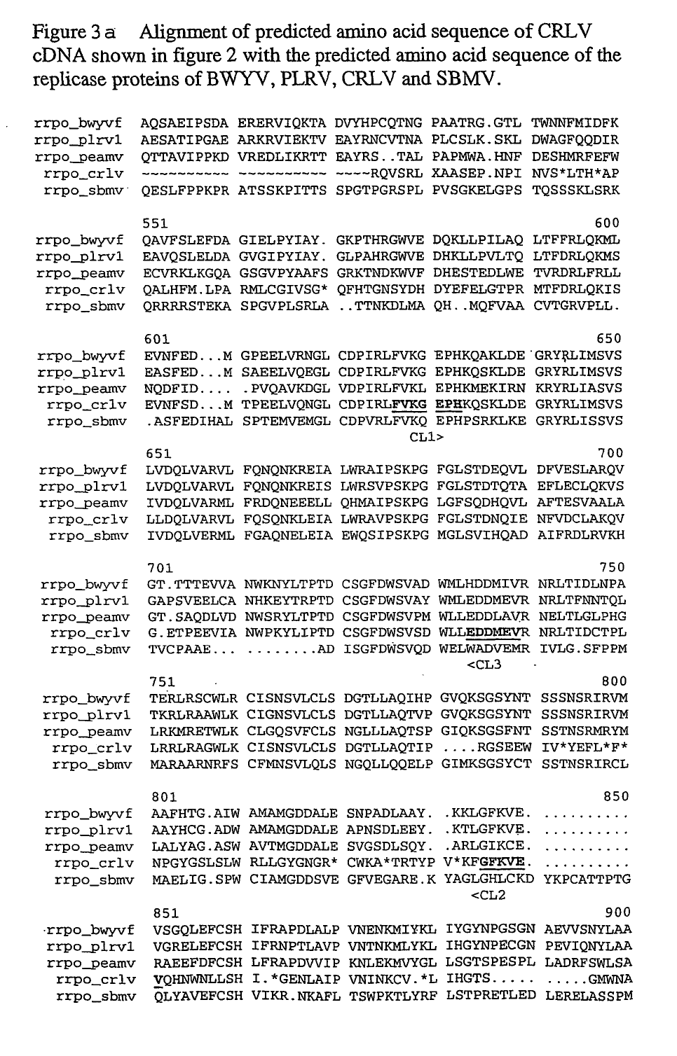

Primers for generating and amplifying cDNAs from CRLV were designed using a previously unpublished partial sequence of CRLV and sequences from BWYV, PLRV, PEMV and SBMV corresponding to the RdRp gene in each of these luteoviruses (Accession nos: P09507, P17520, P29154 and P21405). The CRLV cDNA sequence is shown in FIG. 1 with a partial translation shown in FIG. 2. The translation was aligned with the known RdRp sequences of BWYV, PLRV, PEMV and SBMV using the GCG Wisconsin Package/PileUp software program.

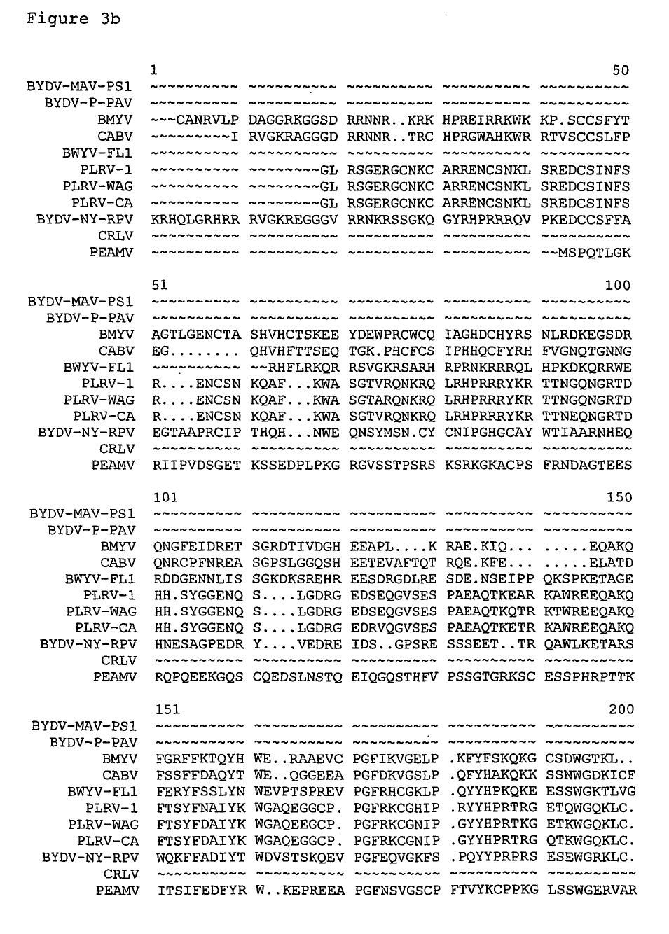

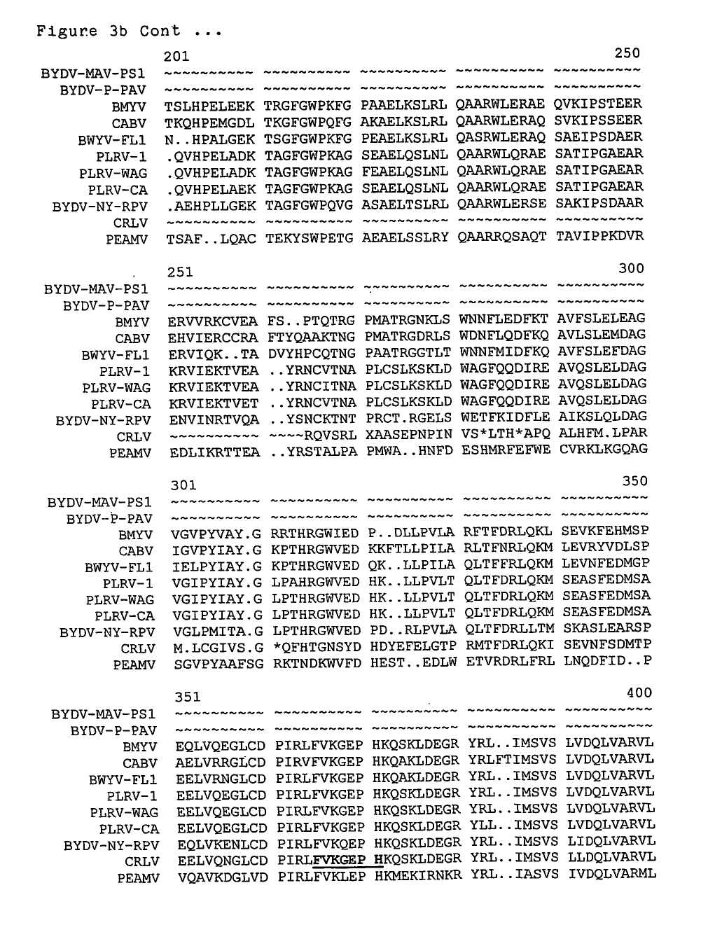

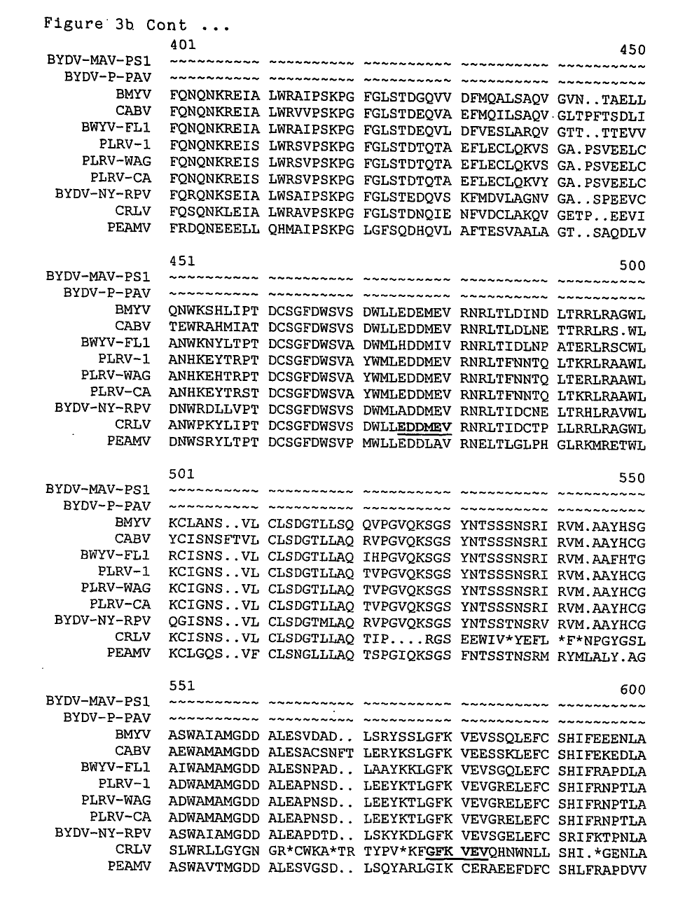

The conserved regions on which the primers were based were chosen specifically as they comprise amino acids with a high degree of degeneracy. The conserved regions identified are shown underlined in FIGS. 3a and 3b. The nucleic acid sequences of the primers are as follows:

| Primer CL1 based on Conserved Region FVKGEPH: | |
|---|---|
| 5′ TTY/GTN/AAR/GGN/GAR/CCN/CAY 3′ | |
| Primer CL2 based on Conserved Region GFKVEV: | |
| 5′ NAC/YTC/NAC/YTT/RAA/NCC 3′ | (antisense and reverse orientation) |
| Primer CL3 based on Conserved Region EDDMEV: | |
| CL3 = 5′ CK/NAC/YTC/CAT/RTC/RTC/YTC 3′ | (antisense and reverse orientation) |

### Example 2—Nucleic Acid Extraction

Approximately 3 g of umbelliferous plant tissue from chervil plants was taken from each sample plant. Total plant RNA was prepared by using an RNeasy Plant Mini Kit (Qiagen). Unless otherwise stated, 3 g leaf material was taken from each plant and placed in a pestle and mortar. Before use, pestles and mortars were soaked in sodium hypochlorite solution, autoclaved and oven-baked to remove ribonucleases and possible traces of contaminating RNA. Liquid nitrogen was added to freeze the leaf material which was then ground to a fine powder. Carrot leaf powder (200 mg) was then added to 450 μl RLC buffer from the kit and total RNA was extracted using a Qi-shredder and an RNeasy column for each sample following manufacturer's instructions. Total RNA was eluted from the final column in 40 μl RNAse free water and stored at −20° C. This procedure gave typical RNA yields of 0.05 μg/ml.

### Example 3—cDNA Synthesis and PCR Primer Annealing

0.5 ml microfuge tubes were used throughout. 100 p mol (1 microlitre) RT-PCR primer (CL2) was added to 1 microlitre plant nucleic acid purified as above. Sterile water was added to 12 microlitres, in each tube.

The mixture was heated at 70° C. for 10 minutes (using a Gene E Techne Thermal cycler PCR block) and quick chilled on ice. 7 microlitres 100× first strand cDNA buffer and 1 microlitre (200 units) of Superscript™ RT enzyme was added. Samples were pulse spun and incubated at 37° C. for 1 hour using a PCR block. (First strand cDNA synthesis buffer stock was made up using 400 microlitres 5× first strand buffer (Gibco BRL), 200 microlitres 0.1 M DTT (Gibco, BRL) 100 microlitres mixed dNTP stock [10 mM each] dATP, dGTP, dCTP and dTTP made up from 100 mM stocks]). Finally, reverse transcriptase was denatured at 95° C. for 8 minutes.

### Example 4—PCR Amplification of cDNA

For luteovirus detection, 5 μl cDNA was added to 95 μl PCR reaction mix [40 μl PCR primer CL1 at 100 pmol/ml, 40 μl PCR primer CL3 at 100 pmol/ml, 400 μl 10× RED Taq PCR Reaction Buffer (Sigma), 8 μl each dNTP (dATP/dGTP/dCTP/dTTP at 100 mM) for 0.2 mM final concentration, sterile water to 4 ml]. 2.5 μl (2.5 U) RED Taq™ DNA polymerase (Sigma) was then added to each tube, and samples were pulse spun and overlaid with a drop of mineral oil. PCR cycling was achieved using a Gene E Techne Thermal cycler PCR block as follows: 95° C. 1 minute denaturing, 45° C. 2 minutes annealing, 72° C. 4 minutes synthesis, for 40 cycles, 1 cycle of 72° C. 10 minute synthesis.

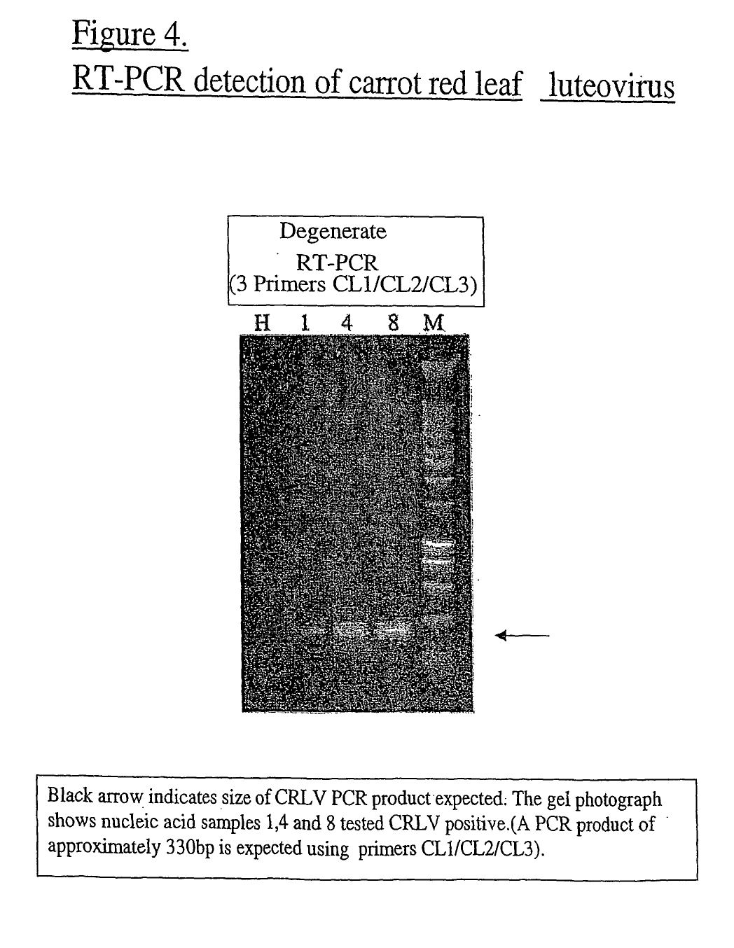

PCR products were analysed by gel electrophoresis. 15 μl PCR reaction and 5 μl gel loading dye were electrophoresed in 1.3% TAE agarose gels containing 0.3 μg/ml ethidium bromide, at 75 volts for 1 hour. DNA molecular weight markers (Bioline) were co-electrophoresed with the PCR products. As shown in FIG. 4 nucleic acid samples 1,4 and 8 tested CRLV positive.

### Example 5—PCR Amplification of cDNA Using CL1 and CL2 as Primers

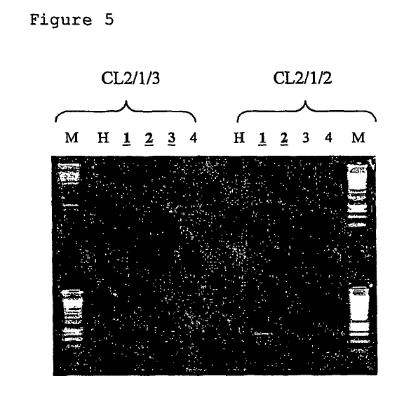

cDNA was amplified as in Example 4, but substituting CL2 for CL3. As with primers CL1/CL2/CL3, a number of nucleic acid samples were found to be positive for CRLV. An example is shown in FIG. 5.

### Example 6—CRLV RT-PCR Sensitivity Assay

A comparison of the sensitivity of the test for CRLV using primers CL2/1/3 (as in Example 4) and CL2/1/2 (as in Example 5) was performed using carrot leaves of wild carrots obtained from a site in Oxfordshire. Nucleic acid was isolated and tested as described for the Chervil leaves above. The results are shown in FIG. 5.

Ten-fold serial dilutions were made of infected carrot leaf RNA with healthy carrot leaf RNA. In FIG. 5, H corresponds to 0.07 mg healthy carrot RNA used in cDNA synthesis with CRLV cDNA primer CL2. Lanes 1-4 correspond to undiluted 0.07 μg infected carrot RNA, ×10 diluted total infected carrot RNA, ×100 (700 pg infected carrot leaf RNA) and ×1000 diluted total infected carrot RNA. CRLV positive samples are shown in bold and underlined. PCR products obtained using three PCR primers are shown on the left and larger PCR products obtained using two PCR primers are shown on the right. A faint PCR band at ×100 dilution is obtained using three different PCR primers in RT-PCR. No PCR product is seen at ×100 dilution using two PCR primers. The results of a repeat dilution series experiment are shown on the lower half of the gel. RT-PCR using CL2 for cDNA synthesis and CL1/CL3 for PCR amplification of cDNA is shown as a more sensitive detection method than RT-PCR using CL2 for cDNA synthesis and CL1/CL2 for PCR amplification of cDNA.

### Example 7—Design and Testing of of Degenerate Primers to Detect CMoV

The predicted polymerase amino acid sequence of the Australian isolate of CMoV (Gibbs, M. J., Cooper, J. I., and Waterhouse, P. M. (1996a).Virology 224:310-313; Gibbs, M. J., Zeigler, A., Robinson, D. J., Waterhouse, P. M., and Cooper, J. I. (1996b). Molecular Plant Pathology On-Line. [http://www.bspp.org.uk/mppol]1996/111gibbs) was aligned with the polymerase region of other carmo-like viruses and regions of amino acid conservation were used to select two degenerate RT-PCR primers (Carmo-2 and Carmo-6). This approach was used to PCR amplify and sequence a region of the polymerase gene of a UK isolate of CMoV. The predicted amino acid sequences of the Australian and UK isolates of CMoV were then aligned and two CMoV specific RT-PCR primers were designed (NC1 and NC2). Carmo-6 was used to prime cDNA synthesis and NC1 and NC2 were used for PCR amplification of cDNA.

The carmovirus related primer Carmo-6 is based on the amino acid sequence VCDDGNN; the deduced nucleic acid sequence is G(AC)(AC)CTGCAG(AGCT)AC(AG)CA(AG)TC(AG)TC(AGCT)CC(AG)TT(AG) TT. Carmovirus related primer Carmo-2 is based on the amino acid sequence V/PRT/VIQP; the deduced nucleic acid sequence AA(AG)GTCGACCCG(AT)(AGCT)CC(AGCT)(AC)G(AGCT)GT(AGCT)AT(ACT)CA ACC.

The carrot mottle specific NC1 and NC2 are based on the amino acid sequences AKGFNAFQ and KYKVKG respectively; the deduced nucleic acid sequences GC(AGCT)AA(AG)GG(AGCT)TT(CT)AATGC(AGCT)TT(CT)C for NC1 and (AG)CA(AGCT)CC(CT)TT(AGCT)AC(CT)TT(AG)TA(CT)TT for NC2.

The first strand cDNA was synthesised as in Example 3, substituting primer Carmo-6 in place of CL2. The cDNA sequence was amplified using the method as described in Example 4, substituting primers NC1 and NC2 in place of primers CL1 and CL3.

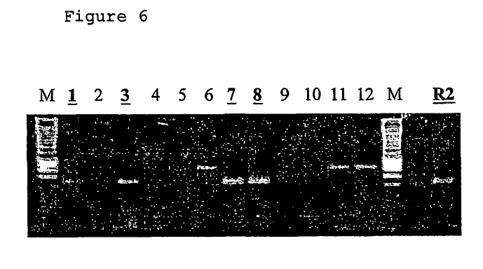

PCR products were analysed as described in Example 4. The results are shown in FIG. 6.

The major CMoV specific PCR product is predicted to be 258 bp long using NC1/NC2 primers in PCR following cDNA synthesis using Carmo-6 primer. CMoV positive samples are shown in bold and underlined (B). The white arrow marked indicates the position of the CMoV PCR product. A number of non-specific PCR bands larger than 258 bp were observed and included a PCR product of approximately 700 bp. A product of this size was seen in healthy controls and is assumed to be non-viral derived. Raising the primer annealing temperature from 45° C. to 47° C. did not remove background host-specific PCR products. Visualisation of degenerate RT-PCR products by gel analysis is therefore critical for determining CMoV positive samples.

1. An oligonucleotide primer capable of hybridising to conserved regions of nucleic acid of carrot red leaf luteovirus (CRLV), potato leafroll polerovirus (PLRV) and at least one of barley yellow dwarf virus (BYDV) and beet western yellow virus (BWYV).

2. A primer according to claim 1 capable of hybridising to conserved regions of nucleic acid of carrot red leaf luteovirus (CRLV), potato leafroll polerovirus (PLRV), barley yellow dwarf virus (BYDV) and beet western yellow virus (BWYV).

3. A primer according to claim 1 or claim 2 wherein the primer hybridises to a region of nucleic acid corresponding to a RNA dependent RNA polymerase (RdRp) gene.

4. The primer according to any one of the preceding claims wherein the primer hybridises to a region of the CRLV sequence of FIG. 1 (SEQ ID NO: 9).

5. The primer according to any one of the preceding claims wherein the primer encodes the amino acid sequence FVKGEPH (SEQ ID NO:1) or KGEPHK (SEQ ID NO:7).

6. The primer according to claim 5 having the nucleotide sequence 5′ TTY/GTN/AAR/GGN/GAR/CCN/CAY 3′ (SEQ ID NO:2) or AAR/GGN/GAR/CCN/CA Y/AAR/C 3′ (CL4, SEQ ID NO:8)

7. The primer according to any one of claims 1 to 5 wherein the primer hybridise to the nucleic acid encoding amino acid sequence GFKVEV (SEQ ID NO:3) or EDDMEV (SEQ ID NO:5).

8. The primer according to claim 8 having the nucleotide sequence 5′NAC/YTC/NAC/YTT/RAA/NCC 3′ (SEQ ID NO:4)) or 5′ CK/NAC/YTC/CAT/RTC/RTC/YTC 3′ (SEQ ID NO:6).

9. A method for diagnosing the presence of a luteovirus sequence in a plant tissue nucleic acid sample, the method comprising the step of: hybridising a primer according to any one of claims 1 to 8 to the nucleic acid.

10. A method according to claim 9 comprising the steps of treating the nucleic acid with a first PCR primer according to claim 1 to 9 to obtain a DNA product; treating the DNA product with one or more second PCR primers according to claim 1 to 9 to obtain a PCR product; and comparing the nucleotide sequence or corresponding amino acid sequence of the PCR product with a known nucleotide sequence or corresponding amino acid sequence characteristic of the luteovirus.

11. A method according to claim 10 wherein said first primer is CL2 (SEQ ID NO:4) and said second primer comprises a mixture of primers CL1 (SEQ ID NO:2) and CL2 (SEQ ID NO:4).

12. A method for diagnosing the presence of at least one of CRLV or PLRV sequences in a plant tissue, the method comprising the steps of:
providing nucleic acid from the plant tissue;
treating the nucleic acid with PCR primer CL2 (SEQ ID NO:4) to obtain a DNA product;
treating the DNA product with PCR primers CL1 (SEQ ID NO:2) and CL3 (SEQ ID NO:6) to obtain a PCR product; and
comparing a nucleotide sequence or corresponding amino acid sequence in the PCR product with a nucleotide sequence or corresponding amino acid sequence characteristic of the luteovirus to detect the presence of the luteovirus.

13. A kit for performing the method of any one of claims 9 to 12 comprising one or more primers according to any one of claims 1 to 8.

14. A kit according to claim 13 further comprising one or more of primers SEQ ID NOS: 12, 14 , 17 or 18.


SEQUENCE LISTING (41 sequences)

SEQ ID NO: 1 - protein, 7 aa, Carrot red leaf luteovirus
FVKGEPH

SEQ ID NO: 2 - DNA, 21 bp, Artificial Sequence
  Description of Artificial Sequence Primer
ttygtnaarggngarccncay

SEQ ID NO: 3 - protein, 6 aa, Carrot red leaf luteovirus
GFKVEV

SEQ ID NO: 4 - DNA, 18 bp, Artificial Sequence
  Description of Artificial Sequence Primer
nacytcnacyttraancc

SEQ ID NO: 5 - protein, 6 aa, Carrot red leaf luteovirus
EDDMEV

SEQ ID NO: 6 - DNA, 20 bp, Artificial Sequence
  Description of Artificial Sequence Primer
cknacytccatrtcrtcytc

SEQ ID NO: 7 - protein, 6 aa, Carrot red leaf luteovirus
KGEPHK

SEQ ID NO: 8 - DNA, 19 bp, Artificial Sequence
  Description of Artificial Sequence Primer
aarggngarccncayaarc

SEQ ID NO: 9 - DNA, 1100 bp, Carrot red leaf luteovirus
  misc_feature (21): n=a or g or c or t/u
cccgacaggtttcccgactgnaagcggccagtgagcccaacccaattaatgtgagttagctcactcattaggcaccccaggctttacactttatgcttccggctcgtatgttgtgtggaattgtgagcggataacaatttcacacaggaaacagctatgaccatgattacgaattcgagctcggtaccccgagaatgacttttgatcgactacagaagatatcagaagtcaatttcagtgatatgacaccggaggagctagttcaaaacgggctctgcgatcctatcagactattcgtcaagggagagccccacaaacagagcaaactcgatgaaggccgctaccgcctcataatgagtgtgtcattactagaccaattggtagcccgggtcttgttccagtctcagaacaagttagaaatcgcgctttggagagcagtaccttcaaaacccggatttggcctttccacagacaatcaaattgaaaattttgtagattgtctagccaagcaagtgggggagacaccagaagaggtcatagctaactggcctaaatacttaatccccacagattgctcaggcttcgattggtcagtttcagactggctcctcgaagatgacatggaggtgagaaatcgcttgaccattgactgcacccctcttttacggaggctgcgagcaggatggttgaaatgcatttccaactctgtcttgtgcctttctgatggcaccctgttagcccaaactatcccccggggttcagaagagtggatcgtataatacgagttcctctaattctagaatccgggttatggcagcctatcattgtggcgcctcttgggctatggcaatgggcgatgatgctggaaagcatagacacggacctatccggtataaaaatttgggtttaaagtcgaggttcagcacaactggaatttgctctcacatatttaaggagagaacctcgccattccggtgaatatcaacaaatgtgtataacttatccacggtacatccggaatgtggaacgcgagtctcagcactattaggcgtgctcgcatgatgaggtcagacgtacacggactaagccaatcgttatgg

SEQ ID NO: 10 - protein, 18 aa, Carrot red leaf luteovirus
  SITE (7): Xaa is uncertain
RQVSRLXAASEPNPINVS

SEQ ID NO: 11 - protein, 7 aa, Carrot mottle umbravirus
VCDDGNN

SEQ ID NO: 12 - DNA, 30 bp, Artificial Sequence
  Description of Artificial Sequence Primer
gmmctgcagnacrcartcrtcnccrttrtt

SEQ ID NO: 13 - protein, 6 aa, Carrot mottle umbravirus
  SITE (1): Xaa is Val or Pro
XRXIQP

SEQ ID NO: 14 - DNA, 31 bp, Artificial Sequence
  Description of Artificial Sequence Primer
aargtcgacccgwnccnmgngtnathcaacc

SEQ ID NO: 15 - protein, 8 aa, Carrot mottle umbravirus
AKGFNAFQ

SEQ ID NO: 16 - protein, 6 aa, Carrot mottle umbravirus
KYKVKG

SEQ ID NO: 17 - DNA, 22 bp, Artificial Sequence
  Description of Artificial Sequence Primer
gcnaarggnttyaatgcnttyc

SEQ ID NO: 18 - DNA, 21 bp, Artificial Sequence
  Description of Artificial Sequence Primer
rcanccyttnacyttrtaytt

SEQ ID NO: 19 - protein, 20 aa, Carrot red leaf luteovirus
APQALHFMLPARMLCGIVSG

SEQ ID NO: 20 - protein, 214 aa, Carrot red leaf luteovirus
QFHTGNSYDHDYEFELGTPRMTFDRLQKISEVNFSDMTPEELVQNGLCDPIRLFVKGEPHKQSKLDEGRYRLIMSVSLLDQLVARVLFQSQNKLEIALWRAVPSKPGFGLSTDNQIENFVDCLAKQVGETPEEVIANWPKYLIPTDCSGFDWSVSDWLLEDDMEVRNRLTIDCTPLLRRLRAGWLKCISNSVLCLSDGTLLAQTIPRGSEEWIV

SEQ ID NO: 21 - protein, 4 aa, Carrot red leaf luteovirus
YEFL

SEQ ID NO: 22 - protein, 19 aa, Carrot red leaf luteovirus
NPGYGSLSLWRLLGYGNGR

SEQ ID NO: 23 - protein, 4 aa, Carrot red leaf luteovirus
CWKA

SEQ ID NO: 24 - protein, 6 aa, Carrot red leaf luteovirus
TRTYPV

SEQ ID NO: 25 - protein, 18 aa, Carrot red leaf luteovirus
KFGFKVEVQHNWNLLSHI

SEQ ID NO: 26 - protein, 14 aa, Carrot red leaf luteovirus
GENLAIPVNINKCV

SEQ ID NO: 27 - protein, 18 aa, Carrot red leaf luteovirus
LIHGTSGMWNASLSTIRR

SEQ ID NO: 28 - protein, 380 aa, Beet western yellows virus
AQSAEIPSDAERERVIQKTADVYHPCQTNGPAATRGGTLTWNNFMIDFKQAVFSLEFDAGIELPYIAYGKPTHRGWVEDQKLLPILAQLTFFRLQKMLEVNFEDMGPEELVRNGLCDPIRLFVKGEPHKQAKLDEGRYRLIMSVSLVDQLVARVLFQNQNKREIALWRAIPSKPGFGLSTDEQVLDFVESLARQVGTTTTEVVANWKNYLTPTDCSGFDWSVADWMLHDDMIVRNRLTIDLNPATERLRSCWLRCISNSVLCLSDGTLLAQIHPGVQKSGSYNTSSSNSRIRVMAAFHTGAIWAMAMGDDALESNPADLAAYKKLGFKVEVSGQLEFCSHIFRAPDLALPVNENKMIYKLIYGYNPGSGNAEVVSNYLAA

SEQ ID NO: 29 - protein, 381 aa, Potato leaf roll virus
AESATIPGAEARKRVIEKTVEAYRNCVTNAPLCSLKSKLDWAGFQQDIREAVQSLELDAGVGIPYIAYGLPAHRGWVEDHKLLPVLTQLTFDRLQKMSEASFEDMSAEELVQEGLCDPIRLFVKGEPHKQSKLDEGRYRLIMSVSLVDQLVARVLFQNQNKREISLWRSVPSKPGFGLSTDTQTAEFLECLQKVSGAPSVEELCANHKEYTRPTDCSGFDWSVAYWMLEDDMEVRNRLTFNNTQLTKRLRAAWLKCIGNSVLCLSDGTLLAQTVPGVQKSGSYNTSSSNSRIRVMAAYHCGADWAMAMGDDALEAPNSDLEEYKTLGFKVEVGRELEFCSHIFRNPTLAVPVNTNKMLYKLIHGYNPECGNPEVIQNYLAA

SEQ ID NO: 30 - protein, 377 aa, Pea enation mosaic virus
QTTAVIPPKDVREDLIKRTTEAYRSTALPAPMWAHNFDESHMRFEFWECVRKLKGQAGSGVPYAAFSGRKTNDKWVFDHESTEDLWETVRDRLFRLLNQDFIDPVQAVKDGLVDPIRLFVKLEPHKMEKIRNKRYRLIASVSIVDQLVARMLFRDQNEEELLQHMAIPSKPGLGFSQDHQVLAFTESVAALAGTSAQDLVDNWSRYLTPTDCSGFDWSVPMWLLEDDLAVRNELTLGLPHGLRKMRETWLKCLGQSVFCLSNGLLLAQTSPGIQKSGSFNTSSTNSRMRYMLALYAGASWAVTMGDDALESVGSDLSQYARLGIKCERAEEFDFCSHLFRAPDVVIPKNLEKMVYGLLSGTSPESPLLADRFSWLSA

SEQ ID NO: 31 - protein, 11 aa, Carrot red leaf luteovirus
LIHGTSGMWNA

SEQ ID NO: 32 - protein, 379 aa, Southern bean mosaic virus
QESLFPPKPRATSSKPITTSSPGTPGRSPLPVSGKELGPSTQSSSKLSRKQRRRRSTEKASPGVPLSRLATTNKDLMAQHMQFVAACVTGRVPLLASFEDIHALSPTEMVEMGLCDPVRLFVKQEPHPSRKLKEGRYRLISSVSIVDQLVERMLFGAQNELEIAEWQSIPSKPGMGLSVIHQADAIFRDLRVKHTVCPAAEADISGFDWSVQDWELWADVEMRIVLGSFPPMMARAARNRFSCFMNSVLQLSNGQLLQQELPGIMKSGSYCTSSTNSRIRCLMAELIGSPWCIAMGDDSVEGFVEGAREKYAGLGHLCKDYKPCATTPTGQLYAVEFCSHVIKRNKAFLTSWPKTLYRFLSTPRETLEDLERELASSPM

SEQ ID NO: 33 - protein, 562 aa, Beet mild yellowing virus
CANRVLPDAGGRKGGSDRRNNRKRKHPREIRRKWKKPSCCSFYTAGTLGENCTASHVHCTSKEEYDEWPRCWCQIAGHDCHYRSNLRDKEGSDRQNGFEIDRETSGRDTIVDGHEEAPLKRAEKIQEQAKQFGRFFKTQYHWERAAEVCPGFIKVGELPKFYFSKQKGCSDWGTKLTSLHPELEEKTRGFGWPKFGPAAELKSLRLQAARWLERAEQVKIPSTEERERVVRKCVEAFSPTQTRGPMATRGNKLSWNNFLEDFKTAVFSLELEAGVGVPYVAYGRRTHRGWIEDPDLLPVLARFTFDRLQKLSEVKFEHMSPEQLVQEGLCDPIRLFVKGEPHKQSKLDEGRYRLIMSVSLVDQLVARVLFQNQNKREIALWRAIPSKPGFGLSTDGQVVDFMQALSAQVGVNTAELLQNWKSHLIPTDCSGFDWSVSDWLLEDEMEVRNRLTLDINDLTRRLRAGWLKCLANSVLCLSDGTLLSQQVPGVQKSGSYNTSSSNSRIRVMAAYHSGASWAIAMGDDALESVDADLSRYSSLGFKVEVSSQLEFCSHIFEEENLA

SEQ ID NO: 34 - protein, 565 aa, CABV
IRVGKRAGGGDRRNNRTRCHPRGWAHKWRRTVSCCSLFPEGQHVHFTTSEQTGKPHCFCSIPHHQCFYRHFVGNQTGNNGQNRCPFNREASGPSLGGQSHEETEVAFTQTRQEKFEELATDFSSFFDAQYTWEQGGEEAPGFDKVGSLPQFYHAKQKKSSNWGDKICFTKQHPEMGDLTKGFGWPQFGAKAELKSLRLQAARWLERAQSVKIPSSEEREHVIERCCRAFTYQAAKTNGPMATRGDRLSWDNFLQDFKQAVLSLEMDAGIGVPYIAYGKPTHRGWVEDKKFTLLPILARLTFNRLQKMLEVRYVDLSPAELVRRGLCDPIRVFVKGEPHKQAKLDEGRYRLFTIMSVSLVDQLVARVLFQNQNKREIALWRVVPSKPGFGLSTDEQVAEFMQILSAQVGLTPFTSDLITEWRAHMIATDCSGFDWSVSDWLLEDDMEVRNRLTLDLNETTRRLRSWLYCISNSFTVLCLSDGTLLAQRVPGVQKSGSYNTSSSNSRIRVMAAYHCGAEWAMAMGDDALESACSNFTLERYKSLGFKVEESSKLEFCSHIFEKEDLA

SEQ ID NO: 35 - protein, 520 aa, Beet western yellows virus
RHFLRKQRRSVGKRSARHRPRNKRRRQLHPKDKQRRWERDDGENNLISSGKDKSREHREESDRGDLRESDENSEIPPQKSPKETAGEFERYFSSLYNWEVPTSPREVPGFRHCGKLPQYYHPKQKEESSWGKTLVGNHPALGEKTSGFGWPKFGPEAELKSLRLQASRWLERAQSAEIPSDAERERVIQKTADVYHPCQTNGPAATRGGTLTWNNFMIDFKQAVFSLEFDAGIELPYIAYGKPTHRGWVEDQKLLPILAQLTFFRLQKMLEVNFEDMGPEELVRNGLCDPIRLFVKGEPHKQAKLDEGRYRLIMSVSLVDQLVARVLFQNQNKREIALWRAIPSKPGFGLSTDEQVLDFVESLARQVGTTTTEVVANWKNYLTPTDCSGFDWSVADWMLHDDMIVRNRLTIDLNPATERLRSCWLRCISNSVLCLSDGTLLAQIHPGVQKSGSYNTSSSNSRIRVMAAFHTGAIWAMAMGDDALESNPADLAAYKKLGFKVEVSGQLEFCSHIFRAPDLA

SEQ ID NO: 36 - protein, 553 aa, Potato leaf roll virus
GLRSGERGCNKCARRENCSNKLSREDCSINFSRENCSNKQAFKWASGTVRQNKRQLRHPRRRYKRTTNGQNGRTDHHSYGGENQSLGDRGEDSEQGVSESPAEAQTKEARKAWREEQAKQFTSYFNAIYKWGAQEGGCPPGFRKCGHIPRYYHPRTRGETQWGQKLCQVHPELADKTAGFGWPKAGSEAELQSLNLQAARWLQRAESATIPGAEARKRVIEKTVEAYRNCVTNAPLCSLKSKLDWAGFQQDIREAVQSLELDAGVGIPYIAYGLPAHRGWVEDHKLLPVLTQLTFDRLQKMSEASFEDMSAEELVQEGLCDPIRLFVKGEPHKQSKLDEGRYRLIMSVSLVDQLVARVLFQNQNKREISLWRSVPSKPGFGLSTDTQTAEFLECLQKVSGAPSVEELCANHKEYTRPTDCSGFDWSVAYWMLEDDMEVRNRLTFNNTQLTKRLRAAWLKCIGNSVLCLSDGTLLAQTVPGVQKSGSYNTSSSNSRIRVMAAYHCGADWAMAMGDDALEAPNSDLEEYKTLGFKVEVGRELEFCSHIFRNPTLA

SEQ ID NO: 37 - protein, 553 aa, Potato leaf roll virus
GLRSGERGCNKCARRENCSNKLSREDCSINFSRENCSNKQAFKWASGTARQNKRQLRHPRRRYKRTTNGQNGRTDHHSYGGENQSLGDRGEDSEQGVSESPAEAQTKQTRKTWREEQAKQFTSYFDAIYKWGAQEEGCPPGFRKCGNIPGYYHPRTKGETKWGQKLCQVHPELADKTAGFGWPKAGFEAELQSLNLQAARWLQRAESATIPGAEARKRVIEKTVEAYRNCITNAPLCSLKSKLDWAGFQQDIREAVQSLELDAGVGIPYIAYGLPTHRGWVEDHKLLPVLTQLTFDRLQKMSEASFEDMSAEELVQEGLCDPIRLFVKGEPHKQSKLDEGRYRLIMSVSLVDQLVARVLFQNQNKREISLWRSVPSKPGFGLSTDTQTAEFLECLQKVSGAPSVEELCANHKEHTRPTDCSGFDWSVAYWMLEDDMEVRNRLTFNNTQLTERLRAAWLKCIGNSVLCLSDGTLLAQTVPGVQKSGSYNTSSSNSRIRVMAAYHCGADWAMAMGDDALEAPNSDLEEYKTLGFKVEVGRELEFCSHIFRNPTLA

SEQ ID NO: 38 - protein, 553 aa, Potato leaf roll virus
GLRSGERGCNKCARRENCSNKLSREDCSINFSRENCSNKQAFKWASGTVRQNKRQLRHPRRRYKRTTNEQNGRTDHHSYGGENQSLGDRGEDRVQGVSESPAEAQTKETRKAWREEQAKQFTSYFDAIYKWGAQEGGCPPGFRKCGNIPGYYHPRTRGQTKWGQKLCQVHPELAEKTAGFGWPKAGSEAELQSLNLQAARWLQRAESATIPGAEARKRVIEKTVETYRNCVTNAPLCSLKSKLDWAGFQQDIREAVQSLELDAGVGIPYIAYGLPTHRGWVEDHKLLPVLTQLTFDRLQKMSEASFEDMSAEELVQEGLCDPIRLFVKGEPHKQSKLDEGRYLLIMSVSLVDQLVARVLFQNQNKREISLWRSVPSKPGFGLSTDTQTAEFLECLQKVYGAPSVEELCANHKEYTRSTDCSGFDWSVAYWMLEDDMEVRNRLTFNNTQLTKRLRAAWLKCIGNSVLCLSDGTLLAQTVPGVQKSGSYNTSSSNSRIRVMAAYHCGADWAMAMGDDALEAPNSDLEEYKTLGFKVEVGRELEFCSHIFRNPTLA

SEQ ID NO: 39 - protein, 570 aa, Barley yellow dwarf virus
KRHQLGRHRRRVGKREGGGVRRNKRSSGKQGYRHPRRRQVPKEDCCSFFAEGTAAPRCIPTHQHNWEQNSYMSNCYCNIPGHGCAYWTIAARNHEQHNESAGPEDRYVEDREIDSGPSRESSSEETTRQAWLKETARSWQKFFADIYTWDVSTSKQEVPGFEQVGKFSPQYYPRPRSESEWGRKLCAEHPLLGEKTAGFGWPQVGASAELTSLRLQAARWLERSESAKIPSDAARENVINRTVQAYSNCKTNTPRCTRGELSWETFKIDFLEAIKSLQLDAGVGLPMITAGLPTHRGWVEDPDRLPVLAQLTFDRLLTMSKASLEARSPEQLVKENLCDPIRLFVKQEPHKQSKLDEGRYRLIMSVSLIDQLVARVLFQRQNKSEIALWSAIPSKPGFGLSTEDQVSKFMDVLAGNVGASPEEVCDNWRDLLVPTDCSGFDWSVSDWMLADDMEVRNRLTIDCNELTRHLRAVWLQGISNSVLCLSDGTMLAQRVPGVQKSGSYNTSSTNSRVRVMAAYHCGASWAIAMGDDALEAPDTDLSKYKDLGFKVEVSGELEFCSRIFKTPNLA

SEQ ID NO: 40 - protein, 5 aa, Carrot red leaf luteovirus
GENLA

SEQ ID NO: 41 - protein, 537 aa, Pea enation mosaic virus
MSPQTLGKRIIPVDSGETKSSEDPLPKGRGVSSTPSRSKSRKGKACPSFRNDAGTEESRQPQEEKGQSCQEDSLNSTQEIQGQSTHFVPSSGTGRKSCESSPHRPTTKITSIFEDFYRWKEPREEAPGFNSVGSCPFTVYKCPPKGLSSWGERVARTSAFLQACTEKYSWPETGAEAELSSLRYQAARRQSAQTTAVIPPKDVREDLIKRTTEAYRSTALPAPMWAHNFDESHMRFEFWECVRKLKGQAGSGVPYAAFSGRKTNDKWVFDHESTEDLWETVRDRLFRLLNQDFIDPVQAVKDGLVDPIRLFVKLEPHKMEKIRNKRYRLIASVSIVDQLVARMLFRDQNEEELLQHMAIPSKPGLGFSQDHQVLAFTESVAALAGTSAQDLVDNWSRYLTPTDCSGFDWSVPMWLLEDDLAVRNELTLGLPHGLRKMRETWLKCLGQSVFCLSNGLLLAQTSPGIQKSGSFNTSSTNSRMRYMLALYAGASWAVTMGDDALESVGSDLSQYARLGIKCERAEEFDFCSHLFRAPDVV